(19) United States
(12) Patent Application Publication
(10) Pub. No.: US 2015/0024043 A1
(43) Pub. Date: Jan. 22, 2015

(54) Medi-Cap (Bio-Absorbable Capsules)

(71) Applicant: Sachin Patel, Diamond Bar, CA (US)

(72) Inventor: Sachin Patel, Diamond Bar, CA (US)

(21) Appl. No.: 13/917,642

(22) Filed: Jul. 18, 2013

(51) Int. Cl. A61K 9/48; A61K 47/46; A61K 47/34

(52) U.S. Cl. CPC A61K 9/4891; A61K 47/34; A61K 47/46
USPC 424/453; 424/451; 424/457

(57) ABSTRACT

Medi-Cap a specialized absorbable capsule of various shapes and sizes. It may come prefilled with desired medication or material or it can come in a two-piece snap together capsule that can be filled with desired medication/material by the medical professional. (Most Likely prefilled) Medi-Cap will be time released meaning that each capsule used in an area of treatment will have a specific time in which the capsule will degrade and release the medication in the area of operation or treatment. Depending on what Medi-Cap will be used for there should be different capsules with different absorbing times.

1.) What is Medi-Cap?

Medi-Cap a specialized absorbable capsule of various shapes and sizes. It may come prefilled with desired medication or material or it can come in a two-piece snap together capsule that can be filled with desired medication/material by the medical professional. (Most Likely prefilled) Medi-Cap will be time released meaning that each capsule used in an area of treatment will have a specific time in which the capsule will degrade and release the medication in the area of operation or treatment. Depending on what Medi-Cap will be used for there should be different capsules with different absorbing times.

2.) What Is Med-Cap Made of?

Vicryl (Polyglactin 910), Chromic gut, catgut, Polyglycolic acid, Polylactic Acid, Polydioxanone or any other type of Bio-Absorbable material that can be degraded by the human body. These capsules will have to be time released depending on what they will be used for which can be layered to form a capsule. Polyglactin (Vicryl), Chromic gut, catgut and the other materials/chemicals listed above are materials used in suturing of open wounds. We know that the body degrades these materials by mainly a process known as Hydrolysis or proteolytic enzymatic degradation. So we do know that Medi-cap can be used in the human body. Medications will need to be formulated by a pharmaceutical company depending on what the capsule will be used for.

3.) Some Applications of Medi-Cap

I would initially like to have these capsules contain Antibiotics for application of deep wounds which would not heal by primary closure and would have to let heal by secondary intention and as well as after major surgery in which these absorbable capsules filled with antibiotics can be place in the wounds before closure to aid in healing and prevent post-surgical infections.

Tooth impaction Surgery (Trans-alveolar surgery) in which a time absorbing capsule of antibiotics may be placed in the mucosa and sutured closed. (Limits oral antibiotics as well as Aids in Healing. Cyst enucleations

I would like to use it in allographic as well as autogenous bone grafts (This will be the empty capsules in which the medical professional will use)

4.) Advantages of Medi-Cap

Can limit the use of oral/I.V/I.M antibiotics and Painkillers depending on a case to case basis.

Cheap to make

BIO-ABSORBABLE

May be used in other applications not mentioned

May have a wide range of uses in medical/dental/veterinary and related fields.

5.) Additional Information

Medi-Cap is designed to especially facilitate the delivery of certain medications, Sizes and Shapes may vary between a ½ inch to 8 inches depending on the area of treatment.

1. I Claim that Medi-Cap will be used in certain fields of Medicine, Dentistry. Veterinary Medicine as well as other branches of the healthcare related fields.
